(19) United States
(12) Patent Application Publication
(10) Pub. No.: US 2023/0309905 A1
(43) Pub. Date: Oct. 5, 2023

(54) SYSTEM AND METHOD FOR USING MEDICAL IMAGING DEVICES TO PERFORM NON-INVASIVE DIAGNOSIS OF A SUBJECT

(71) Applicant: YISSUM RESEARCH DEVELOPMENT COMPANY OF THE HEBREW UNIVERSITY OF JERUSALEM LTD., Jerusalem (IL)

(72) Inventors: Aviv MEZER, Jerusalem (IL); Elior DRORI, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/011,893

(22) PCT Filed: Jun. 22, 2021
(86) PCT No.: PCT/IL2021/050762

§ 371 Date: Dec. 21, 2022

Related U.S. Application Data
(60) Provisional application No. 62/705,329, filed on Jun. 22, 2020.
(60) Provisional application No. 63/168,400, filed on Mar. 31, 2021.

(51) Int. Cl. A61B 5/00; A61B 5/055; G06T 7/11; G06T 7/174; G01R 33/56; G01R 33/563; G01R 33/50

(52) U.S. Cl. CPC A61B 5/4082; A61B 5/055; G06T 7/11; A61B 5/7275; G06T 7/174; G01R 33/5608; G01R 33/5602; G01R 33/56341; G01R 33/50; G06T 2207/10088; G06T 2207/30016

(57) ABSTRACT

A system and method of non-invasive diagnosis of a condition in a subject may include obtaining, from a medical scanning device, a three-dimensional (3D) scan of the subject, said scan comprising a plurality of quantitative or semi-quantitative voxel values; segmenting the scan, to obtain a segmented region of interest (ROI) of the subject; performing a singular value decomposition (SVD) of the ROI, to determine at least one axis of the ROI in the SVD space; and analyzing the voxel values along the at least one axis, to diagnose a condition of the subject. Analysis of the voxel values may include calculating a quantitative function of voxel values along the at least one axis; comparing the calculated quantitative function to a reference quantitative function; and diagnosing or predicting a condition of the subject, based on the comparison.

### CROSS-REFERENCE TO RELATED APPLICATIONS

This application is a PCT International Application claiming the benefit of priority of U.S. Patent Application No. 62/705,329, filed Jun. 22, 2020, and entitled: “MEASURING BIOLOGICAL GRADIENTS ALONG THE HUMAN DORSAL STRIATUM IN VIVO USING QUANTITATIVE MRI”, and of U.S. Patent Application No. 63/168,400, filed Mar. 31, 2021, and entitled “MEASURING BIOLOGICAL GRADIENTS ALONG THE HUMAN DORSAL STRIATUM IN VIVO USING QUANTITATIVE MRI”, which are hereby incorporated by reference in their entirety.

### FIELD OF THE INVENTION

The present invention relates generally to the field of medical imaging. More specifically, the present invention relates to using medical imaging devices to perform non-invasive diagnosis of a subject.

### BACKGROUND OF THE INVENTION

Currently available methods of diagnosis of brain-related conditions in-vivo are typically performed based on non-quantitative (e.g., morphological) MRI mapping or scanning. Such methods are normally limited to detection of gross morphological abnormalities or focal abnormalities resulting in regional differences in signal intensities within the scanned tissue. High-end systems and methods for producing MRI scans, commonly referred to as quantitative MRI scans or mappings may provide raw information representing microstructures of a scanned tissue, however: (a) such high-end systems are normally not available for most health centers and clinics, and (b) automated analysis of quantitative MRI is still a matter of research and study.

### SUMMARY OF THE INVENTION

As elaborated herein, embodiments of the invention are adapted to produce semi-quantitative scans, that may represent microstructural-level information of a scanned tissue, from commonly available weighted scans, as elaborated herein. Additionally, embodiments of the invention may automatically determine regions of interest in the quantitative, or semi-quantitative scans, and automatically analyze these regions of interest to obtain various markers regarding a subject's condition. Such markers may include, for example information regarding a patient's normal, or abnormal aging, a diagnosis of a suspected brain-related disease such as Parkinson's disease, a prognosis of a suspected disease, and the like.

Embodiments of the invention may include a method of non-invasive diagnosis of a condition in a subject by at least one processor. Embodiments of the method may include obtaining, from a medical scanning device, a three-dimensional (3D) scan of the subject. said scan may include a plurality of voxel values; segmenting the scan, to obtain a segmented region of interest (ROI) of the subject; performing a singular value decomposition (SVD) of the ROI, to determine at least one axis of the ROI in the SVD space; and analyzing the voxel values along the at least one axis, to diagnose a condition of the subject, as elaborated herein.

According to some embodiments of the invention, analyzing the voxel values along the at least one axis may include calculating a quantitative function of voxel values along the at least one axis; comparing the calculated quantitative function to a reference quantitative function; and diagnosing a condition of the subject, based on the comparison.

According to some embodiments of the invention, the medical scanning device may be a magnetic resonance imaging (MRI) scanning device. The voxel values may be quantitative, or semi-quantitative voxel values, including for example R1, R2, T1, T2, T1w/T2w, proton density and macromolecular tissue volume (MTV) values, diffusion parameter values selected from MD, FA, QSM and CEST values, and any combination thereof.

For example, the medical scanning device may be an MRI scanning device, and wherein scanning the subject may include obtaining a first, weighted T1 scan of the subject, may include a first plurality of voxel values; obtaining a second, weighted T2 scan of the subject, may include a second plurality of voxel values; and elementwise dividing the first plurality of voxel values by the second plurality of voxel values, to obtain a semi-quantitative voxel values.

The segmented ROI may be, or may include a putamen of the subject. In such embodiments, the quantitative function may represent a spatial variation (or “gradient”, as referred to herein) of voxel values along the at least one axis, through the putamen. Additionally, or alternatively, the segmented ROI may be, or may include a caudate of the subject. In such embodiments, the quantitative function may represent a spatial variation or gradient of voxel values along the at least one axis, through the caudate.

According to some embodiments of the invention, analyzing the voxel values along the at least one axis further may include calculating a quantitative correlation function, that may represent correlation between (a) quantitative values of one or more voxels along the at least one axis of the ROI and (b) quantitative values of one or more voxels located in another region of the subject's brain, such as the cortex. Embodiments of the invention may subsequently compare the calculated correlation function to a reference correlation function (e.g., stored in a repository) and diagnose, prognose or predict a condition of the subject based on the comparison, as elaborated herein.

For example, embodiments of the invention may predict a condition of dopaminergic loss in the subject based on the comparison, as elaborated herein. Additionally, or alternatively, embodiments of the invention may predict a condition of motor function decline in the subject based on the comparison, as elaborated herein.

According to some embodiments of the invention, the calculated quantitative function may include or may be an asymmetry quantitative function that may represent a metric of asymmetry between (a) quantitative voxel values along at least one axis of a left hemisphere striatum and (b) quantitative voxel values along at least one axis of a right hemisphere striatum. Embodiments of the invention may subsequently compare the asymmetry quantitative function to a reference asymmetry quantitative function and predict a condition of dopaminergic loss in the subject based on said comparison. Additionally, or alternatively, embodiments of the invention may compare the asymmetry quantitative function to a reference quantitative function and predict a condition of motor function decline in the subject based on the comparison.

Embodiments of the invention may include a system for non-invasive diagnosis of a condition in a subject. Embodiments of the system may include a non-transitory memory device, wherein modules of instruction code may be stored, and a processor associated with the memory device, and configured to execute the modules of instruction code. Upon execution of said modules of instruction code, the processor may be configured to obtain, from a medical scanning device, a three-dimensional (3D) scan of the subject, said scan may include a plurality of voxel values; segment the scan, to obtain a segmented region of interest (ROI) of the subject; perform a singular value decomposition (SVD) of the ROI, to determine at least one axis of the ROI in the SVD space; and analyze the voxel values along the at least one axis, to diagnose, prognose or predict a condition of the subject.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included within an embodiment of a system for non-invasive diagnosis of a condition in a subject, according to some embodiments;

FIG. 2 is a block diagram, depicting a system for non-invasive diagnosis of a condition in a subject, according to some embodiments;

FIGS. 3A (3A-1 through 3A-6), 3B (3B-1 through 3B-6), 3C (3C-1 through 3C-3), 3D (3D-1, 3D-2) and 3E (3E-1, 3E-2) depict experimental measurements of quantitative values in three different locations of the brain, along a plurality of axes;

FIGS. 4A (4A-1 through 4A-3) and 4B (4B-1, 4B-2) depict experimental measurements of aging-related changes in microstructural gradients of the striatum;

FIGS. 5A-5C depict experimental measurements of various quantitative MRI parameters, and associated spatial and aging-related changes;

FIGS. 6A, 6B (6B-1, 6B-2), 6C (6C-1, 6C-2), 6D, 6E, 6F and 6G depict experimental measurements of spatial variations, or gradients in semi-quantitative (e.g., T1w/T2w) voxel values, obtained from MRI scans of a plurality of subjects;

FIGS. 7A, 7B and 7C are graphs of experimental measurements, depicting microstructural spatial variations or gradients of cortico-striatal covariation; and

FIG. 8 is a flow diagram depicting a method of non-invasive diagnosis of a condition in a subject, according to some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

The dorsal striatum is a major input structure of the basal ganglia, playing a crucial role in motor and cognitive aspects of goal-directed behavior. Changes in the striatal tissue are associated with motor and cognitive dysfunctions that take place in normal aging and in neurodegenerative disorders such as Parkinson's disease (PD). Importantly, the function of the striatum is tightly related to its microstructural properties. Histochemical studies highlight distinct neurochemical compartments of the striatal tissue (e.g., striosomes and matrix), that differ in dopaminergic and cholinergic expression and change in distribution and chemical properties along axes of the striatum. These compartments are thought to constitute an organizational feature of striatal connectivity. Indeed, connectivity of the striatum is linked to cortical hierarchal organization, showing gradients along the anterior-posterior and ventromedial-dorsolateral axes that correspond to sensorimotor, associative, and limbic systems. Accordingly, electrophysiology and fMRI studies provide evidence for functional and cognitive gradients in the striatum.

In this context, the terms “gradient” or “gradient function” may be used herein to refer to manifestation of a spatial change (e.g., a change along a spatial axis) in one or more parameters or quantitative values that characterize a tested tissue.

Parkinson's disease and parkinsonism show gradients of striatal microstructure vulnerability, mainly along the anterior-posterior axis, emphasizing acute degeneration in posterior parts of the putamen. This degeneration is characterized by depletion of dopaminergic innervation to the dorsal striatum, resulting from loss of dopaminergic neurons in the substantia nigra pars compacta in PD patients. Indeed, positron emission tomography (PET) and single photon emission computed tomography (SPECT) studies demonstrate the progression of dopamine intake decreases in the striatum in PD. Striatal deterioration in turn affects motor function, leading to a range of movement impairments. Hence, microstructural gradients in the striatum are key feature in understanding the striatum's function in conditions of health and disease.

Current methods of studying the microstructure of the striatum mainly employ invasive animal research and human postmortem methods. In living humans, it is mostly done invasively, using emission tomography. Magnetic Resonance Imaging (MRI) is the main non-invasive tool for structural research of the human brain. Developments in quantitative MRI (qMRI) provide parametric mappings of biophysical tissue properties (e.g., relaxometry rate R1). There is accumulating evidence that qMRI is sensitive to a variety of biological sources in the microenvironment of the tissue. Such biological sources include, for example, as myelin, water content and iron concentration. Therefore, qMRI may facilitate “in vivo histology” of the human brain.

The term “microstructure” may be used herein to refer to structural, compositional, and/or molecular characteristics of cellular formation of a tissue. For example, microstructure of a brain tissue may refer to a content of water in the tissue, type, quantity and/or concentration of cells in the tissue, content of myelin in the tissue, type, quantity and/or composition of lipids and/or proteins in the tissue, and the like.

Embodiments of the invention may use qMRI to quantify microstructural gradients of the striatum, to determine or diagnose a condition of a subject in vivo. For example, embodiments of the invention may determine an aging of a subject as normal or abnormal, based on the quantified gradients of the striatum. In another example, embodiments of the invention may diagnose a subject as suffering from PD, and/or determine a prognosis of PD in vivo, based on the quantified gradients of the striatum.

As elaborated herein, embodiments of the invention may automatically measure spatial variation in microstructure along main axes of the striatum of a specific subject. Embodiments of the invention may produce one or more spatial functions of microstructural change that may be used for comparison between scans, e.g., between different subjects, between different clinical conditions and/or between different scans of a specific subject.

Embodiments of the invention may characterize striatal gradients in typical healthy younger and older adults from multiple biological sources. Embodiments of the invention may generalize these results, using clinically available semi-quantitative MRI scans, to detect microstructural changes that occur in pathological conditions, such as PD. Embodiments of the invention may further investigate the association of these detected microstructural changes to the pathological (e.g., parkinsonian) symptomatology.

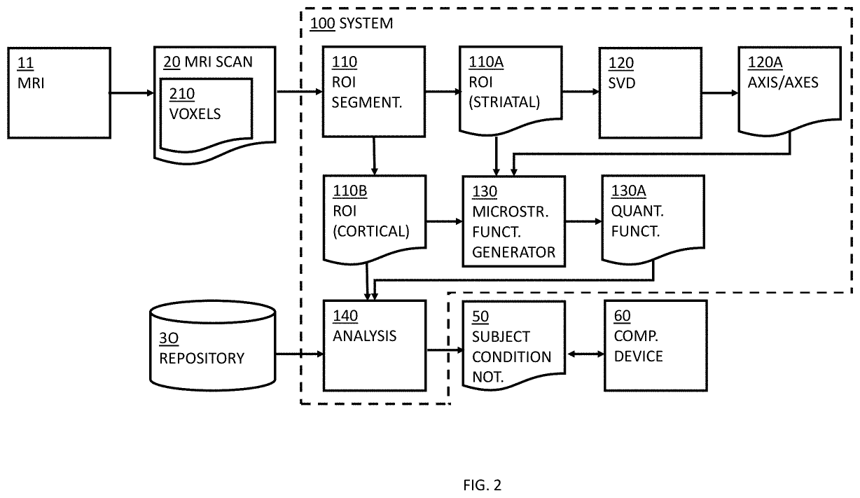

Additionally, in the case of PD, embodiments of the invention may determine a first relation between the detected microstructural changes and changes in dopamine levels, which may, for example, be quantified using PET. As elaborated herein, this first relation may be utilized to diagnose and/or prognose a condition of a subject that is suspected of suffering from PD. For example, and as elaborated herein (e.g., in relation to FIG. 2), embodiments of the invention may produce a subject condition notification that may include information pertaining to diagnosis, or characterization of a human subject (e.g., a patient), monitoring of efficacy of a drug therapy, prognosis of a subject's disease, and the like.

As known in the art, studies have shown correlation between striatal microstructure and cortical networks. Striatal subregions have been correlated with differential cortical connectivity and co-activity. In other words, striatal structure has been shown to be connected with cortical function. Embodiments of the invention may characterize a second relation between the detected microstructural changes and cortical activity. As elaborated herein, this second relation may be utilized to diagnose and/or prognose a condition of a subject that is suspected of suffering from various brain-related pathologies.

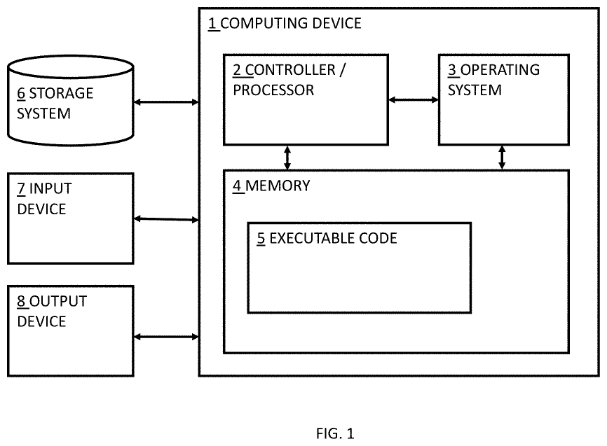

Reference is now made to FIG. 1, which is a block diagram depicting a computing device, which may be included within an embodiment of a system for non-invasive diagnosis of a condition in a subject, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may perform non-invasive diagnosis of a condition in a subject, as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data obtained from medical scanning devices (e.g., quantitative MRI scans, PET scans etc.) may be stored in storage system 6 and may be loaded from storage system 6 into memory 4, where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPUs) or any other suitable multi-purpose or specific processors or controllers (e.g., similar to element 2), a plurality of input units, a plurality of output units, a plurality of memory units, and a plurality of storage units.

Reference is now made to FIG. 2, which depicts a system for non-invasive diagnosis of a condition in a subject, according to some embodiments.

According to some embodiments of the invention, system 100 may be implemented as a software module, a hardware module, or any combination thereof. For example, system 100 may be, or may include a computing device such as element 1 of FIG. 1, and may be adapted to execute one or more modules of executable code (e.g., element 5 of FIG. 1) to perform non-invasive diagnosis of a condition in a subject, as further described herein. As shown in FIG. 2, arrows may represent flow of one or more data elements to and from system 100 and/or among modules or elements of system 100. Some arrows have been omitted in FIG. 2 for the purpose of clarity.

As known in the art, postmortem studies have found spatial microstructural gradients along axes of the striatum. According to some embodiments of the invention, system 100 may be configured to determine the spatial variability in microstructure along axes of the human dorsal striatum in vivo, and in a non-invasive manner, as elaborated herein. Additionally, system 100 may produce a notification of a subject condition, based on the determined spatial variability, as elaborated herein. This notification is denoted herein as “subject condition notification 50”, or “notification 50” for short. Notification 50 may include for example information pertaining to diagnosis and/or assessment of a subject's condition, based on the determined spatial variability, as elaborated herein.

According to some embodiments, system 100 may be communicatively connected (e.g., via a communication network, such as the Internet) to an MRI device 11, configured to produce a three-dimensional (3D) quantitative or semi-quantitative MRI scan 20 (or “scan 20”, for short) of a subject, such as a human patient.

Scan 20 may include a plurality of quantitative, or semi-quantitative voxel values 210. The terms “quantitative value”, “quantitative parameter” and “quantitative scan” may be used herein interchangeably. These terms may be used herein to indicate data that has been acquired through an MRI scan, and includes numerical values, that may be attributed to specific voxels or locations of the scanned tissue. Quantitative values may represent measurable physical or chemical variables, and may be expressed in physical units, to compare between tissue regions and among subjects. As known in the art, by this definition, most clinical MRI acquisitions are not quantitative. Traditionally, clinical MRI relies on the acquisition of so-called “weighted images”, whose image contrast is affected by a combination of different factors, some intrinsic to the tissue and some dependent on the specifics of the scanning procedure. Application of traditional (e.g., non-quantitative) scans is normally limited to revealing either gross morphological abnormalities or focal abnormalities resulting in regional differences in signal intensities within the scanned tissue. In other words, non-quantitative scans are intrinsically insensitive to subtle global changes that may affect the entire scanned tissue.

For example, quantitative scanning voxel values 210 may include R1 values or T1 values, which may represent a combination of Iron concentration and Myelin concentration lipid and macromolecules composition in the scanned tissue, and may thus be used to evaluate a level of tissue microstructure, as known in the art. In another example, quantitative scanning voxel values 210 may include R2* values and/or QSM values, which may represent iron concentration, iron proteins form and Myelin in the scanned tissue, as known in the art. In another example, quantitative scanning voxel values 210 may include macromolecular tissue volume (MTV) values, which may be associated with the non-water content of the scanned tissue, as known in the art. In another example, quantitative scanning voxel values 210 may include proton density values, which may be associated with the water content of the scanned tissue, as known in the art. In another example, quantitative scanning voxel values 210 may include R2 values or T2 values, which may be associated with Iron concentration and Myelin concentration lipid and macromolecules composition in the scanned tissue, as known in the art. In another example, quantitative scanning voxel values 210 may include diffusion parameter values, such as MD, FA may be associated with myelin concentration and cellular and axonal organization and CEST, which may be associated with and myelin concentration lipid and macromolecules composition.

The term “semi-quantitative” may be used herein to refer to voxel values that may be processed so as to represent measurable physical or chemical properties of the scanned tissue. For example, as known in the art, T1w and T2w are non-quantitative weighted voxel values that may normally be used for representing morphological aspects in an MRI image. However, a combination of T1w and T2w, e.g., pointwise division of T1w and T2w (e.g., T1w/T2w) has been experimentally found to represent a combination of Iron concentration and Myelin concentration in the scanned tissue, and may therefore substitute R1 values, to evaluate a level of tissue microstructure. In another example, semi-quantitative voxel values may be, or may include ratio of other weighted image voxel values, such as a magnetization transfer ratio (MTR) that includes a magnetization transfer (MT) weighted image and a non-MT weighted image. MTR has been experimentally found to represent a combination of myelin concentration in the scanned tissue, and may therefore substitute R1 values, to evaluate a level of tissue microstructure. In another example, semi-quantitative voxel values may be, or may include other weighted image voxel values such as R2*W and PD*W. Additional types of semi-quantitative voxel values as known in the art may also be used.

According to some embodiments, system 100 may receive or obtain scan 20 (including quantitative, or semi-quantitative scanning voxel values 210) from MRI device 11. System 100 may include a region of interest (ROI) segmentation module 110, configured to segment scan 20, to obtain a segmented ROI 110A, 110B of the scanned tissue of the subject.

In some embodiments, segmentation module 110 may be, or may include a machine learning (ML) based model, adapted to segment a predefined region of MRI scan 20 to obtain ROI 110A/110B, by any appropriate image segmentation algorithm, as known in the art. ROI 110A/110B may be, or may include a data structure such as a table or a 3D array that may represent, or mask a location that defines the predefined region.

For example, ROI 110A may be a data structure that may include indices of voxels 210 that define a location of a striatum in MRI scan 20. In another example, ROI 110A may be a data structure that may include indices of voxels 210 that define a location of a portion of a striatum (e.g., a putamen, a caudate) in MRI scan 20. In another example, ROI 110B may be a data structure that may include indices of voxels 210 that define a location of cerebral cortex in MRI scan 20.

As elaborated herein, system 100 may be configured to automatically assess spatial variability in microstructure along axes of an ROI such as a human dorsal striatum in vivo.

According to some embodiments, system 100 may include a singular value decomposition (SVD) module 120, adapted to perform SVD on region of interest 110A, to automatically determine the main axes 120A of the ROI in the SVD space, as known in the art. In other words, SVD module 120 may perform, for each ROI (e.g., each predefined brain structure) and for each scan (e.g., for each subject) a singular value decomposition on the voxel coordinates 210, to find at least one of the main orthogonal axes 120A of ROI 110A.

It may be appreciated that in the putamen and caudate ROIs 110A, the three main axes 120A roughly correspond to the anterior-posterior (AP), ventral-dorsal (VD) and medial-lateral (MLA) axes of the brain. Therefore, the terms AP, VD and MLA may be used herein simplistically, to refer to axes 120A that may be detected by SVD model 120.

According to some embodiments, system 100 may include a microstructure function generator module 130, adapted to calculate or produce a quantitative function 130A (or “function 130A” for short) of voxel values 210, along at least one of the main orthogonal axes 120A of ROI 110A. The term “quantitative function” may be used herein in a sense that function 130A may map a location of a voxel 210 along at least one orthogonal axis 120A to quantitative data that corresponds to a quantitative value of one or more voxels 210 in or around that location.

For example, function 130A may be, or may include a gradient function, representing a difference in quantitative values between evenly spaced locations along at least one axis 120A. In this context, the terms “gradient” or “gradient function” may be used herein to refer to a quantitative function 130A that may represent a change in quantitative values along at least one axis 120A.

According to some embodiments, system 100 may include an analysis module 140, adapted to analyze the voxel values 210 along the at least one axis 120A, to diagnose a condition of the subject, and subsequently produce a subject condition notification 50, as elaborated herein. System 100 may subsequently send or communicate (e.g., via the internet) subject condition notification 50, e.g., as a textual message or email, to a computing device 60 of a relevant user, such as a computing device 60 of a physician or a care giver.

According to some embodiments, analysis module 140 may compare the calculated quantitative function 130A of a tested subject to a reference quantitative function 130A, and may diagnose, or determine a condition of the tested subject, based on this comparison.

In this context, the calculated quantitative function 130A may be said to be compared to a reference quantitative function 130A in a sense that corresponding values of these functions along one or more axes 120A may be compared. Thus, anomalies or outliers (e.g., beyond a predefined threshold) in the values of the calculated quantitative function 130A, in relation to values of reference quantitative function 130A may be found, and may indicate a medical condition of interest.

Pertaining to the example of the gradient quantitative function 130A, analysis module 140 may compare values of a gradient quantitative function 130A to one or more reference gradient quantitative function values 130A that may be stored in a repository database 30. Analysis module 140 may subsequently determine a condition of a relevant subject (e.g., patient) based on that comparison. For example, analysis module 140 may determine whether a patient may be diagnosed with a specific pathological condition, such as PD. Additionally, or alternatively, analysis module 140 may determine whether a subject is presenting normal brain aging, or whether scan 20 represents a condition of abnormal, or accelerated aging of brain tissue.

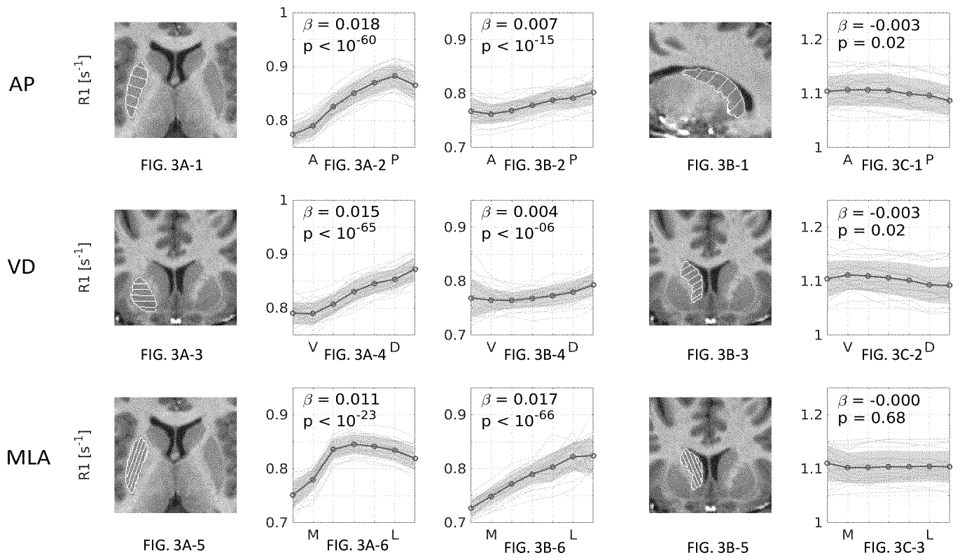

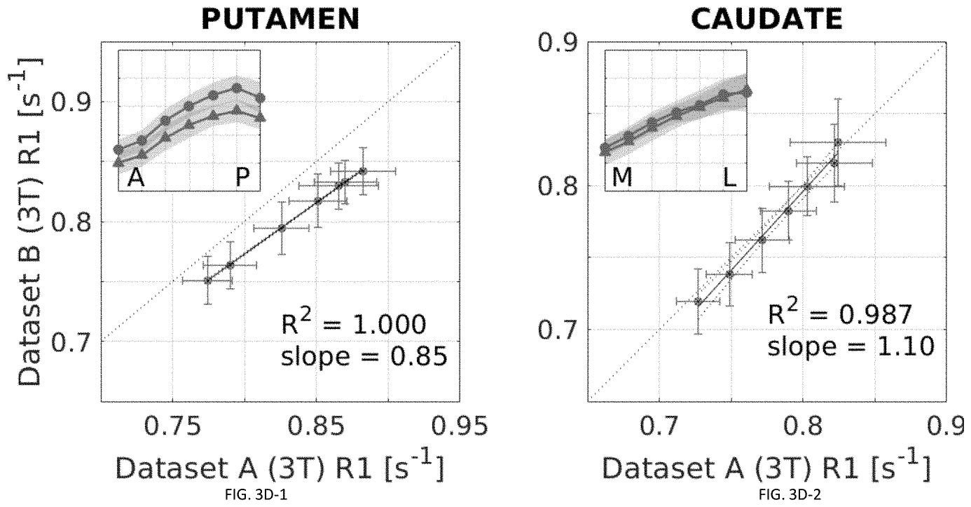

Reference is now made to FIGS. 3A (3A-1 through 3A-6), 3B (3B-1 through 3B-6), 3C (3C-1 through 3C-3), 3D (3D-1, 3D-2) and 3E (3E-1, 3E-2), which depict experimental measurements of quantitative values (e.g., R1 quantitative voxel values 210), in three different locations of the brain, along a plurality of axes.

FIG. 3A-1 depicts a transverse-plane section of a subject's MRI scan 20, where a putamen of the subject is highlighted or segmented. As shown in FIG. 3A-1, the putamen region is divided along the AP axis 120A to a predefined number (e.g., 7, in this example) of regions.

FIG. 3A-2 is a graph, depicting a plurality of measurements corresponding to a plurality of MRI scans 20 along the AP axis 120A of the putamen. As shown in FIG. 3A-2, voxels values 210 (e.g., quantitative R1 values) of scan 20 are classified or attributed to seven equally spaced segments along the AP axis. A median value of voxels values 210 is then computed for each segment, yielding a quantitative function 130A along the AP axis. In other words, in this example, FIG. 3A-2 may depict a quantitative function 130A, where each point represent a median value of voxels values 210, in a segment along the AP axis of the Putamen. The shaded area of the graph in FIG. 3A-2 represents the mean value ±1 standard deviation (STD) value.

FIG. 3A-3 depicts a coronal-plane section of a subject's MRI scan 20, where a putamen of the subject is highlighted or segmented. As shown in FIG. 3A-3, the putamen region is divided along the VD axis 120A to a predefined number (e.g., 7) of regions.

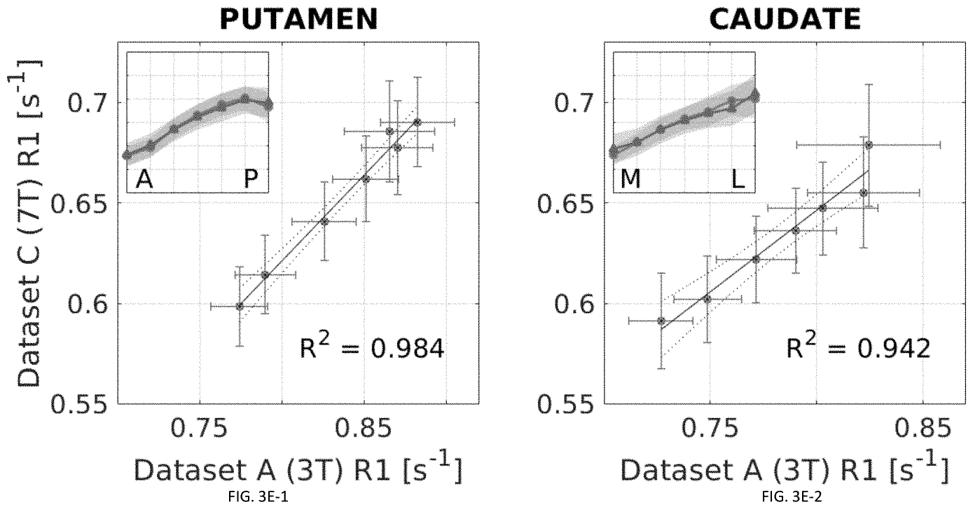

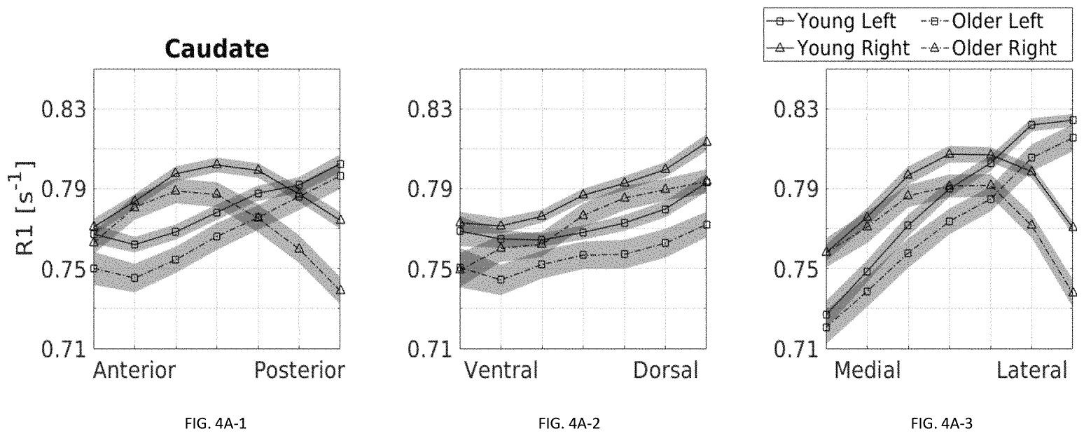

FIG. 3A-4 is a graph, depicting a plurality of measurements corresponding to a plurality of MRI scans 20 along the VD axis 120A of the putamen. It may be appreciated that the graph of quantitative function 130A as depicted in FIG. 3A-4 has been obtained in a similar manner to that of FIG. 3A-2 (relating to the AP axis 120A), and will not be repeated here for the purpose of brevity.

FIG. 3A-5 depicts a transverse-plane section of a subject's MRI scan 20, where a putamen of the subject is highlighted or segmented. As shown in FIG. 3A-5, the putamen region is divided along the MLA axis 120A to a predefined number (e.g., 7) of regions.

FIG. 3A-6 is a graph, depicting a plurality of measurements corresponding to a plurality of MRI scans 20 along the MLA axis 120A of the putamen. It may be appreciated that the graph of quantitative function 130A as depicted in FIG. 3A-6 has been obtained in a similar manner to that of FIG. 3A-2 (relating to the AP axis 120A), and will not be repeated here for the purpose of brevity.

FIG. 3B-1 depicts a sagittal-plane section of a subject's MRI scan 20, where a caudate of the subject is highlighted or segmented. As shown in FIG. 3B-1, the Caudate region is divided along the AP axis 120A to a predefined number (e.g., 7, in this example) of regions.

FIG. 3B-2 is a graph, depicting a plurality of measurements corresponding to a plurality of MRI scans 20 along the AP axis 120A. As shown in FIG. 3B-2, voxels values 210 (e.g., quantitative R1 values) of scan 20 are classified or attributed to the seven equally spaced segments along the AP axis of the caudate. A median value of voxels values 210 is then computed for each segment, yielding a quantitative function along the AP axis. In other words, in this example, FIG. 3B-2 may depict a quantitative function 130A, where each point represents a median value of voxels values 210, in a segment along the AP axis of the caudate. The shaded area of the graph in FIG. 3B-2 may represent the mean value ±1 STD value.

FIG. 3B-3 depicts a coronal-plane section of a subject's MRI scan 20, where a caudate of the subject is highlighted or segmented. As shown in FIG. 3B-3, the caudate region is divided along the VD axis 120A to a predefined number (e.g., 7) of regions.

FIG. 3B-4 is a graph, depicting a plurality of measurements corresponding to a plurality of MRI scans 20 along the VD axis 120A of the caudate. It may be appreciated that the graph of quantitative function 130A as depicted FIG. 3B-4 has been obtained in a similar manner to that of FIG. 3B-2 (relating to the AP axis 120A), and will not be repeated here for the purpose of brevity.

FIG. 3B-5 depicts a transverse-plane section of a subject's MRI scan 20, where a caudate of the subject is highlighted or segmented. As shown in FIG. 3B-5, the caudate region is divided along the MLA axis 120A to a predefined number (e.g., 7) of regions.

FIG. 3B-6 is a graph, depicting a plurality of measurements corresponding to a plurality of MRI scans 20 along the MLA axis 120A of the caudate. It may be appreciated that the graph of quantitative function 130A as depicted in FIG. 3B-6 has been obtained in a similar manner to that of FIG. 3B-2 (relating to the AP axis 120A), and will not be repeated here for the purpose of brevity.

FIGS. 3C-1, 3C-2 and 3C-3 represent quantitative functions 130A, where each point represents a median value of quantitative voxels values 210, in a segment of a white brain matter area that roughly matches the size and shape of the putamen, along the AP, VD and MLA axes, respectively.

As shown herein (e.g., in relation to FIGS. 3D (3D-1, 3D-2) and 3E (3E-1, 3E-2)), experimental results have shown consistency of the quantitative functions 130A over different datasets, and different types of MRI scans.

For example, FIG. 3D-1 is a graph depicting replication of gradient quantitative functions 130A of the putamen, along the AP axis 120A (e.g., as depicted in FIG. 3A-2) in two independent 3-Tesla (3T) datasets (denoted dataset ‘A’ and dataset ‘B’). As shown in FIG. 3D-1, a linear correlation exists between average values of corresponding segments in the two datasets, taken from the AP axes 120A of ROI 110A. Adjusted R2 and p-value of the linear fit have been calculated as presented in the graph, showing solid correlation.

In another example, FIG. 3D-2 is a graph depicting replication of gradient quantitative functions 130A of the caudate, along the MLA axis 120A (e.g., as depicted in FIG. 3B-6) between datasets A and B. As shown in FIG. 3D-2, a linear correlation exists between average values of corresponding segments in the two datasets, taken from the MLA axes 120A of ROI 110A. Adjusted R2 and p-value of the linear fit have been calculated as presented in the graph, showing solid correlation between the datasets.

In another example, FIG. 3E-1 is a graph depicting replication of gradient quantitative functions 130A of the putamen, along the AP axis 120A (e.g., as depicted in FIG. 3A-2) in two independent datasets (denoted dataset ‘A’ and dataset ‘C’). Dataset ‘A’ was obtained by 3-Tesla (3T) MRI scans, and Dataset ‘C’ was obtained by 7-Tesla (7T) scans. As shown in FIG. 3E-1, a linear correlation exists between average values of corresponding segments in the two datasets, taken from the AP axes 120A of ROI 110A. Adjusted R2 and p-value of the linear fit have been calculated as presented in the graph, showing solid correlation between datasets A and C.

In yet another example, FIG. 3E-2 is a graph depicting replication of gradient quantitative functions 130A of the caudate, along the MLA axis 120A (e.g., as depicted in FIG. 3B-6) between datasets A and C. As shown in FIG. 3E-2, a linear correlation exists between average values of corresponding segments in the two datasets, taken from the MLA axes 120A of ROI 110A. Adjusted R2 and p-value of the linear fit have been calculated as presented in the graph, showing solid correlation between the datasets.

By comparing FIGS. 3A (3A-1 through 3A-6), 3B (3B-1 through 3B-6) and 3C (3C-1 through 3C-3), it may be appreciated that spatial changes in the values of quantitative voxels 210 (e.g., as presented by respective quantitative function 130A) may be observed along main axes of the putamen and caudate, but not along axes of the white matter area.

It may be appreciated that the experimental results depicted in FIGS. 3A (3A-1 through 3A-6), 3B (3B-1 through 3B-6), 3C (3C-1 through 3C-3), 3D (3D-1, 3D-2) and 3E (3E-1, 3E-2) indicate significant spatial gradients of R1 along axes of the putamen and caudate, with high replicability across healthy young adult subjects.

To rule out a potential irrelevant source of the measured variability (e.g., MRI field inhomogeneities), experiments have tested for a spatial change in a nearby white-matter region of a similar shape. No change has been found in the quantitative voxel values 210 along any of the axes 120A of this control white-matter region, as shown in FIGS. 3C-1, 3C-2 and 3C-3. Additionally, as depicted in FIGS. 3D (3D-1, 3D-2) and 3E (3E-1, 3E-2), the results were replicated on two additional independent 3T and 7T datasets. Hence, it may be determined that quantitative R1 MRI scans 20 clearly reveal spatial gradients of microstructure in the dorsal striatum (e.g., in the caudate and/or putamen).

Reference is now made to FIGS. 4A (4A-1 through 4A-3) through 4B (4B-1, 4B-2), which depict experimental measurements of aging-related changes in microstructural gradients of the striatum.

As known in the art, normal aging often involves a decline in motor skills, that is associated with changes in the striatum.

The experimental measurements depicted in FIGS. 4A (4A-1 through 4A-3) through 4B (4B-1, 4B-2) relate to two age groups. A first group includes young adults (aged 27±2 years). Results pertaining to this first group are depicted in solid lines. A second group includes older adults (aged 67±6 years). Results pertaining to this first group are depicted in broken lines.

FIGS. 4A-1, 4A-2 and 4A-3 depict quantitative functions 130A in the striatum (e.g., in both hemispheres of the caudate) of both younger and older groups, along the AP, VD and MLA axes 120A. The shaded region in each graph may represent the mean value ±1 STD value.

As shown in FIGS. 4A-1, 4A-2 and 4A-3 the calculated quantitative function 130A values (e.g., mean R1 quantitative values) along the main axes (e.g., AP, VD and MLA, respectively) are visibly lower in the second (older) group of subjects in relation to the first (younger) group of subjects. In other words, FIGS. 4A-1, 4A-2 and 4A-3 depict an aging-related change in the gradient of microstructure of the caudate. This is evident in all axes (e.g., AP, VD and MLA) of the caudate.

As elaborated herein, analysis module 140 may analyze the voxel values along the at least one axis, to diagnose a condition of the subject. Relating to the example FIGS. 4A-1, 4A-2 and 4A-3, analysis module 140 may compare values of a calculated quantitative function 130A (e.g., mean R1 quantitative values) along the main axes (e.g., AP, VD and/or MLA, as depicted in FIGS. 4A-1, 4A-2 and 4A-3) between subjects. For example, analysis module 140 may compare values of a calculated quantitative function 130A that pertains to a tested subject, with corresponding values of calculated quantitative functions of other (e.g., healthy) subjects of different ages that may be stored on repository 30. Analysis module 140 may thus determine whether the tested subject is experiencing normal aging, or whether they may be experiencing exceeded aging, that may be manifested in reduced tissue microstructure. In such embodiments, subject condition notification 50 may, for example, include a notification of a normal aging process, or a warning that an exceeded process of aging was observed in the tested subject. System 100 may subsequently send or communicate (e.g., via the internet) subject condition notification 50, e.g., as a textual message, voice message, email and the like, to a computing device 60 of a relevant user, such as a computing device 60 of a physician or a care giver.

Previous studies have reported interhemispheric differences in volume and/or morphology of the caudate and have suggested observed increase of this interhemispheric asymmetry as a marker of aging.

According to some embodiments of the invention, microstructure function generator module 130 may measure interhemispheric asymmetry and/or enhancement of interhemispheric asymmetry that may not be limited to morphological properties, but also relate to the relevant tissue microstructure. In other words, quantitative function 130A may include or represent information pertaining to interhemispheric asymmetry of morphological tissue structure, along one or more of axes 120A.

FIG. 4B-1 depicts a quantitative function 130A that is an absolute interhemispheric asymmetry observed for the AP axis 120A of the caudate, averaged across young (left) and older (right) groups of adults. As shown in FIG. 4B-1, quantitative function 130A may express interhemispheric asymmetry as within-subject mean absolute difference (MAD) between the left-hemisphere and right-hemisphere of R1 values.

FIG. 4B-2 depicts a quantitative function 130A that is a signed difference between the left-hemisphere and right-hemisphere of R1 quantitative values (e.g., left hemisphere value minus right hemisphere value, (L-R)), along the AP axis 120A.

As shown in FIG. 4B-2, the interhemispheric asymmetry may increase substantially in the older group (broken line) in relation to the younger group (solid line) along the AP axis of the caudate. It may be observed that the aging-related interhemispheric asymmetry may be increased in (a) the anterior caudate (left side), where R1 is generally higher in the right caudate, as well as in (b) the posterior caudate (right side), where R1 is higher in the left caudate. In other words, an interaction of age, hemisphere, and position along the AP axis of the caudate may demonstrate aging-related increase in interhemispheric microstructural asymmetry of the caudate.

As elaborated herein, analysis module 140 may analyze the voxel values along the at least one axis, to diagnose a condition of the subject. Relating to the example FIGS. 4B-1 and/or 4B-2, analysis module 140 may compare values of a calculated quantitative function 130A (e.g., signed and/or absolute difference between the left-hemisphere and right-hemisphere of R1 values) as depicted in FIGS. 4B-1 and/or FIG. 4B-2, between subjects. For example, analysis module 140 may compare values of a calculated quantitative function 130A of interhemispheric asymmetry that pertains to a tested subject (e.g., a patient), with corresponding values of calculated quantitative functions 130A of other (e.g., healthy) subjects of different ages, that may be stored on repository 30. Analysis module 140 may thus determine whether the tested subject is experiencing normal aging, or whether they may be experiencing exceeded aging, that may be manifested in reduced tissue microstructure. In such embodiments, subject condition notification 50 may, for example, include a notification of a normal aging process, or a warning that an exceeded process of aging was observed in the tested subject. System 100 may subsequently send or communicate (e.g., via the internet) subject condition notification 50, e.g., as a textual message, voice message, email, and the like, to a computing device 60 of a relevant user, such as a computing device 60 of a tested subject (e.g., a patient), a computing device 60 of a physician and/or a care giver.

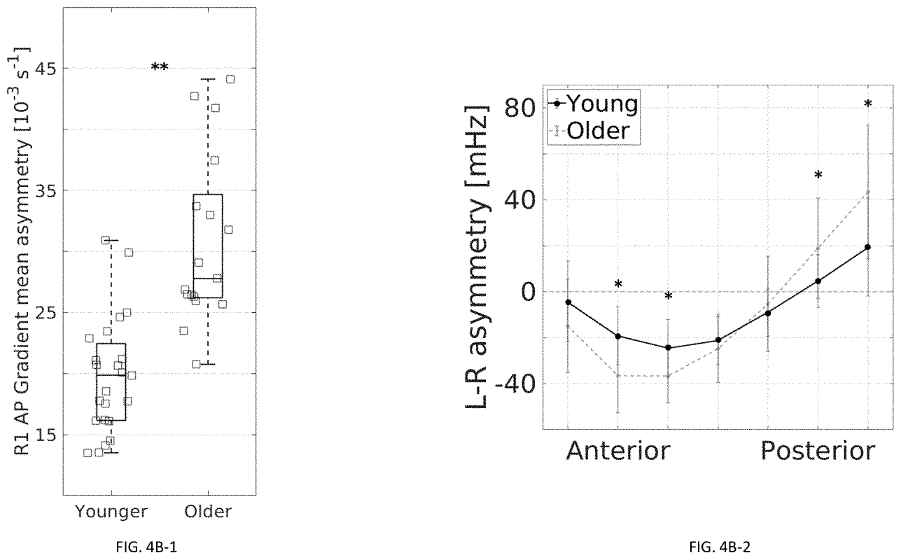

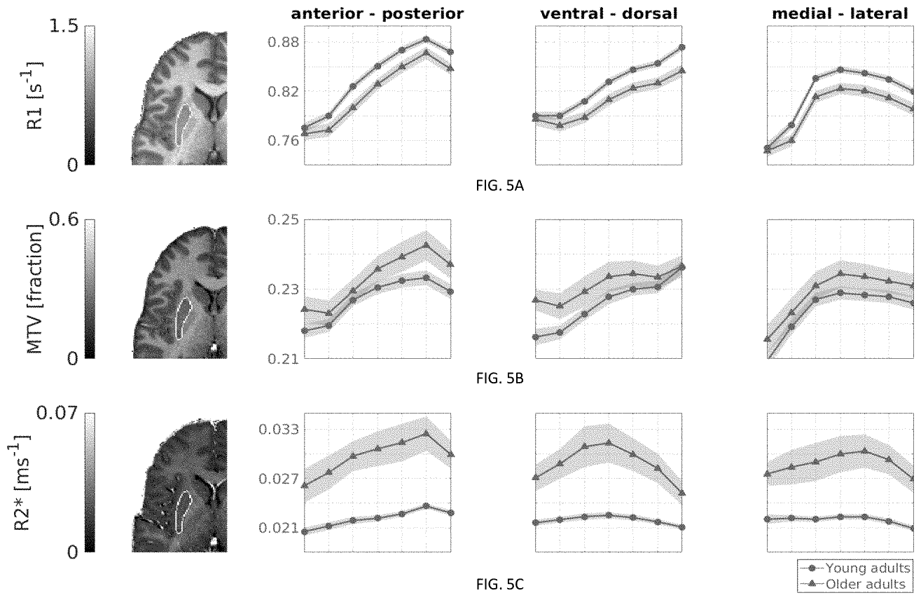

Reference is now made to FIGS. 5A-5C which depict experimental measurements of various quantitative MRI parameters, and associated spatial and aging-related changes. FIG. 5A represents an R1 quantitative scan; FIG. 5B represents an MTV quantitative scan; and FIG. 5C represents an R2* quantitative scan.

Each of FIGS. 5A, 5B and 5C includes, on the left side, a representative axial slice of the respective scan of a single subject, where the putamen is outlined.

Each of FIGS. 5A, 5B and 5C also includes graphs of three calculated quantitative functions 130A, representing spatial variability profiles of the respective quantitative MRI parameter (e.g., R1, MTV and R2*, respectively). The three calculated quantitative functions 130A are plotted along the AP axis 120A (left), VD axis 120A (middle) and MLA axis 120A (right) of the putamen.

Each of the graphs includes two plots. A first plot (circles) shows a mean value of quantitative scans taken from a first group of young subjects. A second plot (triangles) shows a mean value of quantitative scans taken from a second group of older subjects. The shaded area in each plot represents a margin of ±1 Standard Error of Mean (SEM).

According to some embodiments, system 100 may analyze spatial effects of pathological conditions such as PD on the striatal microstructure in vivo and may produce a subject condition notification 50 based on this analysis. In such embodiments, subject condition notification 50 may include, for example, a diagnosis of a pathological condition (e.g., PD), a prognosis of the pathological condition, monitoring of reaction to pharmaceutical treatment, and the like.

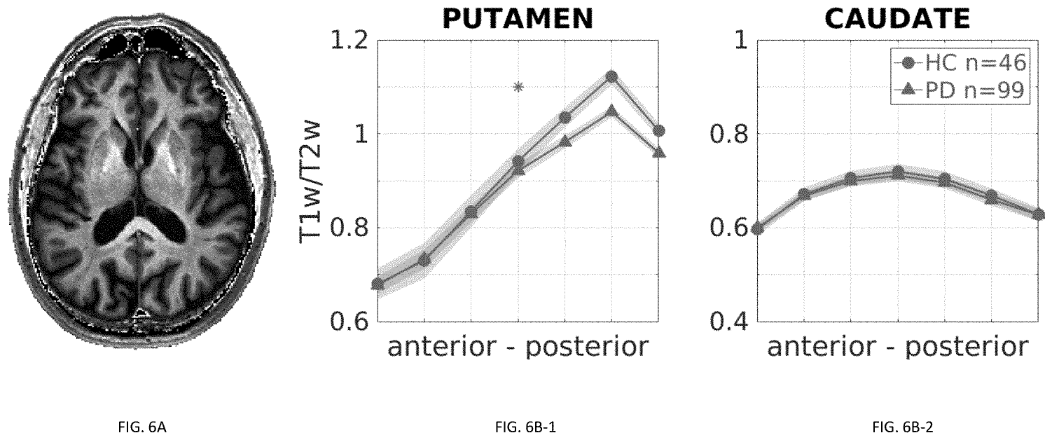

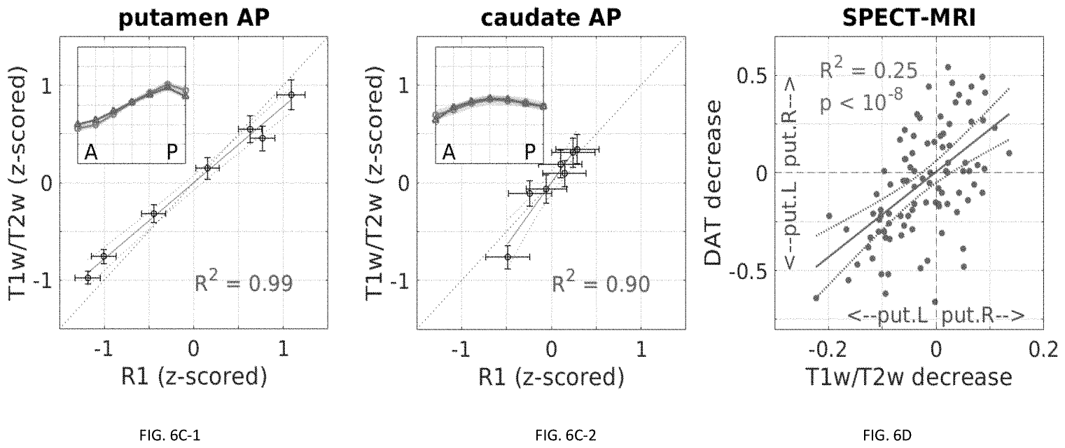

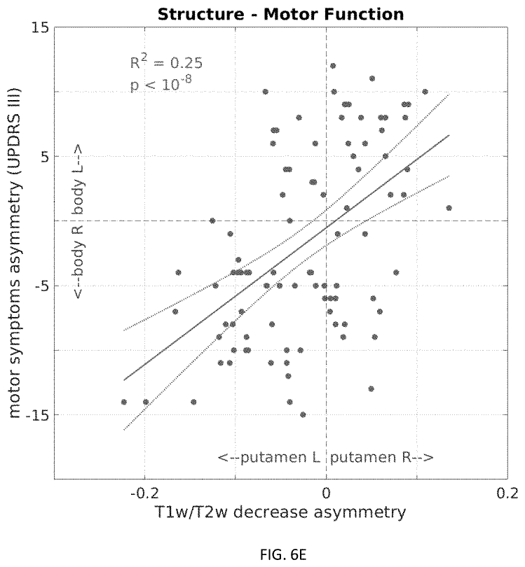

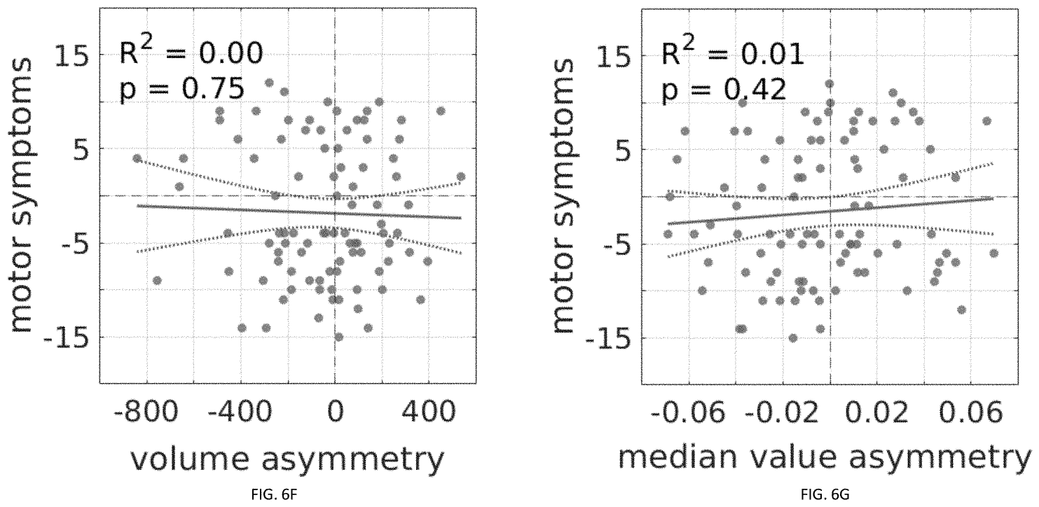

Reference is now made to FIGS. 6A, 6B (6B-1, 6B-2), 6C (6C-1, 6C-2), 6D, 6E, 6F and 6G, which are graphs of experimental measurements, presenting spatial variations or gradients in semi-quantitative (e.g., T1w/T2w) voxel values, obtained from MRI scans 20 of a plurality of subjects.

As elaborated herein, and as depicted in FIGS. 6A, 6B (6B-1, 6B-2), 6C (6C-1, 6C-2), 6D, 6E, 6F and 6G, quantitative functions 130A (e.g., gradient quantitative functions) of quantitative (e.g., R1) or semi-quantitative (e.g., T1w/T2w) voxel values 210 may correspond to microstructure decrease in PD. Embodiments of the invention may analyze these quantitative functions 130A, for example to predict motor deficits of PD patients.

FIG. 6A depicts a representative semi-quantitative (e.g., T1w/T2w) axial slice of an MRI scan 20, pertaining to a 70 year-old male, that had been diagnosed as a PD patient. The MRI scan 20 was obtained from the Parkinson's Progression Marker Initiative (PPMI) T1-weighted and T2-weighted images.

FIGS. 6B (6B-1, 6B-2) depicts semi-quantitative (e.g., T1w/T2w) spatial variation, or gradient quantitative functions 130A, shown for older PD patients (N=99, triangles) and matched (e.g., of a similar age) healthy subjects of a control group (N=46, circles), in the AP axes of the putamen and caudate. The shaded area represents mean±1 STD value. As shown in FIG. 6B (6B-1, 6B-2), the spatial variation or gradient differs between the two groups in the AP axis of the putamen, showing a decrease in posterior subregions in PD.

As shown herein (e.g., in relation to FIGS. 6C-1 and 6C-2), experimental results have shown consistency of the quantitative functions 130A over different datasets. For example, FIG. 6C-1 is a graph depicting replication of gradient quantitative functions 130A of the putamen, along the AP axis 120A (e.g., as depicted in FIG. 6B-1) between two independent datasets: a first dataset pertains to semi-quantitative (e.g., T1w/T2w) scans, and a second dataset pertains to quantitative R1 gradient results. In another example, FIG. 6C-2 is a graph depicting replication of gradient quantitative functions 130A of the caudate along the AP axis 120A (e.g., as depicted in FIG. 6B-2) between the two independent datasets (e.g., T1w/T2w and R1). As shown in FIGS. 6C-1 and 6C-2, a linear correlation exists between average values of corresponding segments in the two datasets, taken from the AP axes 120A of ROI 110A. Adjusted R2 value of the linear fit have been calculated as presented in the graph, showing solid correlation.

FIG. 6D is a linear regression graph, depicting a positive linear correlation between microstructural decrease in the posterior putamen and ipsilateral dopamine decrease. Each point of FIG. 6D represents a single subject (e.g., patient).

The ‘X’ axis of FIG. 6D represents a value of a microstructure asymmetry metric, individually calculated for each subject. In this example, the microstructure asymmetry metric is calculated as the difference between (a) the mean value of quantitative voxels 210 in the left posterior putamen and (b) the mean value of quantitative voxels 210 in the right posterior putamen. Thus, a positive value on the ‘X’ axis of FIG. 6D represents a higher average value in the left posterior putamen, and a negative value on the ‘X’ axis of FIG. 6D represents a higher average value in the right posterior putamen.

The ‘Y’ axis of FIG. 6D represents a value of a dopamine asymmetry metric. The dopamine asymmetry metric is calculated as the difference between (a) the concentration of dopamine in the left putamen, and (b) the concentration of dopamine in the left putamen. Thus, a positive value on the ‘Y’ axis of FIG. 6D represents a higher concentration of dopamine may represent a higher concentration of dopamine in the left putamen, and vise-versa. It may be appreciated that the concentration of dopamine may be obtained, for example by a Single-photon emission computed tomography (SPECT) scan, as known in the art.

According to some embodiments of the invention, microstructure function generator module 130 may calculate a quantitative function 130A of voxel values along at least one axis 120A of the striatum. Analysis module 140 may compare the calculated quantitative function 130A to a reference quantitative function, that may be stored e.g., in repository 30. Analysis module 140 may subsequently predict a condition of dopaminergic loss in the subject based on this comparison, as elaborated herein.

As shown in FIG. 6D, experimental results have established a linear, positive correlation between lateral quantitative, or semi-quantitative (e.g., T1w/T2w) voxel values 210, representing lateral microstructural values, and lateral levels of dopamine. In other words, experimental results have shown that decreased semi-quantitative (e.g., T1w/T2w) voxel values 210 in a first hemisphere (e.g., left) in relation to the other hemisphere (e.g., right) may correlate to decreased dopamine values in the first hemisphere in relation to the other hemisphere. According to some embodiments, system 100 may exploit this linear, positive correlation to diagnose or assess a condition of dopaminergic loss in the subject. Additionally, or alternatively, system 100 may exploit the linear, positive correlation of semi-quantitative (e.g., T1w/T2w) voxel values and lateral of dopamine to predict or anticipate a future condition of dopaminergic loss in the subject.

For example, in some embodiments microstructure function generator 130 calculate or produce a quantitative function 130A which may represent a metric of asymmetry (e.g., a microstructure asymmetry metric, as elaborated herein) between (a) quantitative voxel values 210 along at least one axis 120A of a left hemisphere striatum and (b) quantitative voxel values 210 along at least one axis 120A of a right hemisphere striatum. Analysis module 140 may compare the quantitative function (e.g., representing the microstructure asymmetry metric) to a reference quantitative function 130A, which may be stored on a repository database 30 (e.g., as depicted in FIG. 6D). Analysis module 140 may subsequently produce a subject condition notification 50 that may be, or may include a prediction, diagnosis, assessment and/or prognosis of a condition of dopaminergic loss in the subject based on this comparison. Analysis module 140 may send or transmit (e.g., as an email) subject condition notification 50 to a computing device 60 (e.g., a computing device of a physician), as assistive diagnosis information.

According to some embodiments of the invention, microstructure function generator module 130 may calculate a quantitative function 130A of voxel values along at least one axis 120A of the striatum. Analysis module 140 may compare the calculated quantitative function 130A to a reference quantitative function, that may be stored e.g., in repository 30. Analysis module 140 may subsequently predict a condition of motor function loss in the subject based on this comparison, as elaborated herein.

FIG. 6E is a linear regression graph, depicting a positive linear correlation between a motor symptom laterality score, as elaborated herein, and semi-quantitative (e.g., T1w/T2w) interhemispheric asymmetry of a spatial variation, or gradient quantitative function 130A, in the posterior putamen. Each point of FIG. 6E represents a single subject (e.g., patient).

The ‘X’ axis of FIG. 6E represents a value of a microstructure asymmetry metric, individually calculated for each subject. The microstructure asymmetry metric is calculated as elaborated herein, in relation to FIG. 6D, and will not be repeated for the purpose of brevity.

The ‘Y’ axis of FIG. 6E represents a motor symptom laterality score, which may be calculated based on Unified Parkinson's Disease Rating Scale (UPDRS, part 3) scoring. As known in the art, UPDRS part 3 scoring may relate to specific hemispheres, and may represent a level of decrease in motor functions (e.g., a high score representing extensive decrease in motor functions) pertaining to the relevant hemisphere. In the example of FIG. 6E, the motor symptom laterality score may be calculated as a difference between (a) the left-side (right hemisphere) UPDRS part 3 scoring, and (b) the right-side (left hemisphere) UPDRS part 3 scoring. In other words, a positive value of the motor symptom laterality score may represent a decrease in motor functions that is more extensive in the left side of a subject's body, and vise-versa.

As shown in FIG. 6E, experimental results have shown a positive linear correlation between the motor symptom laterality score and semi-quantitative (e.g., T1w/T2w) interhemispheric asymmetry of a spatial variation or gradient quantitative function 130A. In other words, a decrease in semi-quantitative (e.g., T1w/T2w) levels in the putamen is correlated with decreased motor functions in the left side of the body, and vise-versa. According to some embodiments, system 100 may exploit this linear, positive correlation to diagnose or assess a condition of motor function decline in the subject. Additionally, or alternatively, system 100 may exploit the linear, positive correlation of semi-quantitative (e.g., T1w/T2w) voxel values and decreased motor functions to predict or anticipate a future condition of decline in motor functions in the subject.

For example, in some embodiments microstructure function generator 130 calculate or produce a quantitative function 130A which may represent a metric of asymmetry, such as the microstructure asymmetry metric as elaborated herein, between (a) quantitative voxel values 210 along at least one axis 120A of a left hemisphere striatum and (b) quantitative voxel values 210 along at least one axis 120A of a right hemisphere striatum. Analysis module 140 may compare the quantitative function (e.g., representing the microstructure asymmetry metric) to a reference quantitative function 130A, which may be stored on a repository database 30 (e.g., as depicted in FIG. 6E). Analysis module 140 may subsequently produce a subject condition notification 50 that may be, or may include a prediction, diagnosis, assessment and/or prognosis of a condition of motor function decline in the subject based on this comparison. Analysis module 140 may send or transmit (e.g., as an email) subject condition notification 50 to a computing device 60 (e.g., a computing device of a physician), as assistive diagnosis information.

FIG. 6F is a linear regression graph, showing lack of correlation between symptom laterality and whole-putamen volumetric asymmetry. This lack of correlation is manifested by low Pearson adjusted R2 and high uncorrected p-values. Each point of FIG. 6F represents a single subject (e.g., patient). The ‘X’ axis of FIG. 6F represents a lateral volumetric difference metric. In the example of FIG. 6F, the lateral volumetric difference metric may be calculated as a difference between (a) the volume of the left putamen and (b) the volume of the left putamen. The ‘Y’ axis of FIG. 6F represents a motor symptom laterality score. This motor symptom laterality score may be calculated in a similar manner as elaborated in relation to FIG. 6E, and will not be repeated for the purpose of brevity.

By viewing FIG. 6F it may be appreciated that currently available methods of diagnosis, which may be based on morphological (e.g., volumetric) lateral comparison between the left putamen and right putamen may not be able to correctly predict, or account for symptoms of motor function decrease.

FIG. 6G is a linear regression graph, showing lack of correlation between symptom laterality and whole-putamen median semi-quantitative (e.g., T1w/T2w) asymmetry (e.g., without calculating a gradient quantitative function 130A as depicted in FIG. 6C (6C-1, 6C-2)), according to Pearson adjusted R2 and uncorrected p-values.

The ‘X’ axis of FIG. 6G represents a whole-putamen median semi-quantitative (e.g., T1w/T2w) asymmetry metric. In this example the whole-putamen median T1w/T2w asymmetry metric may be computed as a difference between (a) the median of T1w/T2w voxel values 210 in the left putamen, and (b) the median of T1w/T2w voxel values 210 in the right putamen. The ‘Y’ axis of FIG. 6G represents a motor symptom laterality score. This motor symptom laterality score may be calculated in a similar manner as elaborated in relation to FIG. 6E, and will not be repeated for the purpose of brevity.

By viewing FIG. 6G it may be appreciated that currently available methods of diagnosis, which may be based on lateral comparison of a single quantitative value between the left putamen and right putamen (e.g., without calculating a gradient quantitative function 130A as depicted in FIG. 6C (6C-1, 6C-2)), may not be able to correctly predict, or account for symptoms of motor function decrease.

The Experiments depicted in FIGS. 6A, 6B (6B-1, 6B-2), 6C (6C-1, 6C-2), 6D, 6E, 6F and 6G have analyzed structural MRI data of PD subjects (N=99, aged 65±6 years, 32 female) and matched controls (N=46, aged 65±6 years, 17 female) from PPMI. In these experiments, quantitative R1 voxel values 210 were not available, and semi-quantitative voxel values 210 were used instead: For each subject, a first, T1-weighted scan 20 and a second, T2-weighted scan 20 were obtained from MRI device 11. Subsequently, T1-weighted divided by T2-weighted (T1w/T2w) semi-quantitative voxel values 210 were calculated from the first scan 20 and the second scan 20. As known in the art, the semi-quantitative T1w/T2w ratio may have a contrast that is similar to the quantitative R1 parameter, and is relatively clean from MR-related bias. Thus, the T1w/T2w ratio is commonly used as a semi-quantitative measurement for microstructure.

In other words, and according to some embodiments, system 100 may obtain semi-quantitative (e.g., T1w/T2w) voxel values 210 by obtaining a first, weighted T1 MRI scan 20 of the subject, that includes a first plurality of weighted T1 voxel values 210; obtaining a second, weighted T2 MRI scan 20 of the subject, that includes a second plurality of weighted T2 voxel values; and elementwise dividing the first plurality of voxel values 210 by the second plurality of voxel values 210, to obtain the semi-quantitative (e.g., T1w/T2w) voxel values 210.

According to some embodiments, the segmented striatal ROI 110A may include a putamen of a subject (e.g., a human patient). In such embodiments, quantitative function 130A may be or may represent spatial variation (e.g., a gradient) of voxel values 210 along at least one axis 120A through the putamen. Additionally, or alternatively, the segmented striatal ROI 110A may include a caudate of the subject. In such embodiments, quantitative function 130A may be or may represent a spatial variation (e.g., gradient) of voxel values 210 along the at least one axis 120A, through the caudate.

As shown in FIG. 6B (6B-1, 6B-2), in the striatum of both PD patients and healthy subjects, experiments have found semi-quantitative (e.g., T1w/T2w) gradients with trends that are similar to those quantified by R1, and particularly so in the putamen. This result was later replicated in another experiment, using an independent dataset of the Human Connectome Project (HCP).

In most axes of the dorsal striatum, experiments have found that the semi-quantitative (e.g., T1w/T2w) gradients did not separate the group of PD patients from the control group. However, and as depicted in FIG. 6C (6C-1, 6C-2), experiments have shown a divergence between the PD group and the control group in the AP axis of the putamen. Specifically, mean semi-quantitative (e.g., T1w/T2w) values in the posterior segments of the putamen were decreased in the PD group, in relation to the control group. A mixed-effects model revealed a significant interaction between the position along the AP axis of the putamen and the clinical group, suggesting a small but significant decrease in semi-quantitative (e.g., T1w/T2w) values in the posterior putamen in PD (p-uncorrected=0.003; pFDR <0.05). It may be appreciated that this result agrees with human and non-human primate studies in Parkinson's disease and parkinsonism, which consistently found alterations in the posterior parts of the putamen.

The experiments depicted in FIGS. 6A, 6B (6B-1, 6B-2), 6C (6C-1, 6C-2), 6D, 6E, 6F and 6G further inquired the hypothesis that these microstructural changes were associated with PD-related dopamine loss. Since dopamine loss in the striatum typically manifests asymmetrically, it was hypothesized that in the posterior putamen a greater semi-quantitative value (e.g., T1w/T2w) decrease in one hemisphere would correlate with greater ipsilateral decrease of dopamine transporter (DAT) SPECT binding ratio. As shown in FIG. 6D, experiments have found a strong positive linear correlation between the semi-quantitative value (e.g., T1w/T2w) asymmetry score in the posterior putamen and the DAT standardized binding ratio asymmetry in the putamen (R2=0.25, p<10-8). In other words, experiments have shown that asymmetry in PD-related dopamine decrease is associated with higher ipsilateral microstructural degeneration.

The experiments depicted in FIGS. 6A, 6B (6B-1, 6B-2), 6C (6C-1, 6C-2), 6D, 6E, 6F and 6G further investigated the relationship between the microstructural asymmetry in the posterior putamen, and the laterality in motor symptoms assessed through UPDRS part 3. As shown in FIG. 6E, experiments have found linear correlation between the microstructural asymmetry and motor symptoms laterality, such that a decrease in microstructure in the posterior putamen was associated with higher symptom laterality to the contralateral side of the body (R²=0.25, p<10-8). This effect remained highly significant even if asymmetry was measured considering the whole AP axis of the putamen and not just the posterior part (R²=0.14, p-uncorrected <10-4). In the caudate however, we found no such relation (R²=0.03, p=0.09).

Importantly, in contrast to spatial variation or gradient analysis of quantitative, or semi-quantitative voxel values 210, coarse mean statistics such as the whole-ROI volume (e.g., as depicted in FIG. 6F, R²=0) or whole-ROI semi-quantitative (e.g., T1w/T2w) median value (e.g., as depicted in FIG. 6G, R²=0.01) did not reveal this relation. Furthermore, volumetric asymmetry in the posterior part of the putamen also did not show this correlation (R²=0.02, p=0.16).

Hence, it may be appreciated that a quantitative function 130A representing spatial variation (e.g., a gradient quantitative function 130A) of quantitative (e.g., R1) or semi-quantitative (e.g., T1w/T2w) voxel values 210 may uncover spatial information in the microstructure of the striatum that is associated with: (a) dopamine loss; and/or (b) manifestation of motor deficiencies in Parkinson's disease. According to some embodiments, analysis module 140 may analyze quantitative function 130A, (e.g., a gradient quantitative function 130A representing spatial variation along axis 120A, as depicted in FIG. 6C (6C-1, 6C-2)) as elaborated herein (e.g., in relation to FIGS. 6A, 6B (6B-1, 6B-2), 6C (6C-1, 6C-2), 6D and 6E). Analysis module 140 may subsequently produce a subject condition notification 50, based on this analysis.

For example, in a condition that a tested subject (e.g., a human patient) is suspected as suffering from PD, subject condition notification 50 may include an indication of diagnosis of PD. In another example, in a condition that a tested subject is positively diagnosed (e.g., by a physician) as suffering from PD, subject condition notification 50 may include a prediction and/or assessment of dopamine loss and/or manifestation of motor deficiencies in the tested subject.

As known in the art, relationship between structure and function of the striatum, and the consequent functional deficits in PD, are likely to be mediated by basal ganglia relationship to cortical regions. Postmortem and in-vivo fMRI studies have found connectivity and functional correspondence between striatal subregions and the cortex.

It has been previously hypothesized that spatial variation or gradients of connectivity among regions of the brain may be reflected in variations in the similarity of microstructural properties between these regions. Embodiments of the invention may include analysis of covariance between microstructural properties of the striatum (e.g., putamen and caudate subregions) and other regions in the brain (e.g., cortical regions). As elaborated herein, based on this covariance analysis, embodiments of the invention may: (a) determine which regions (e.g., cortical regions) are structurally associated with the putamen and caudate subregions, and (b) to what extent are the identified regions associated with the putamen and caudate subregions.

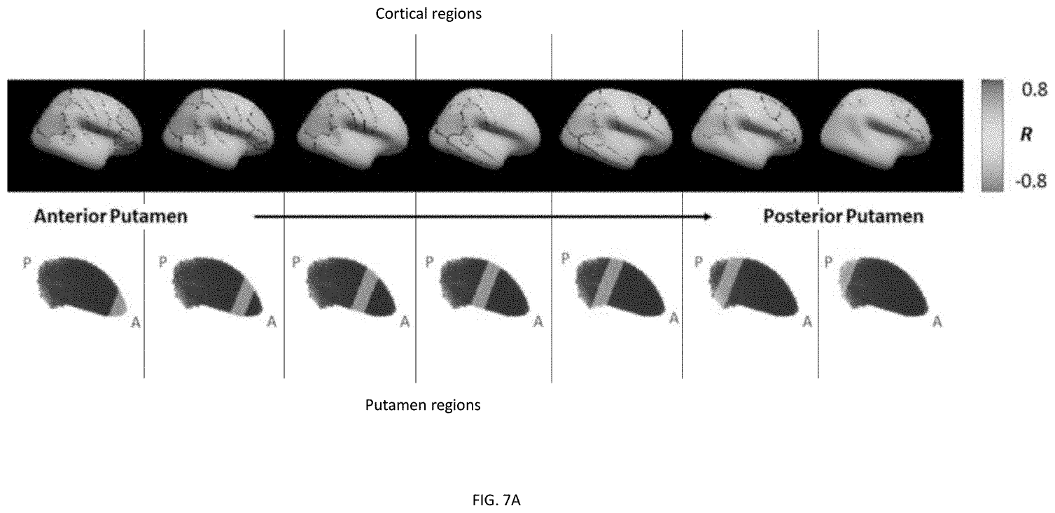

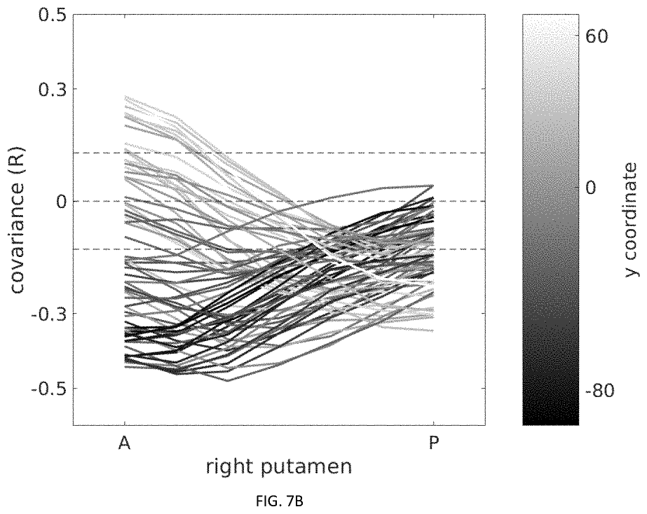

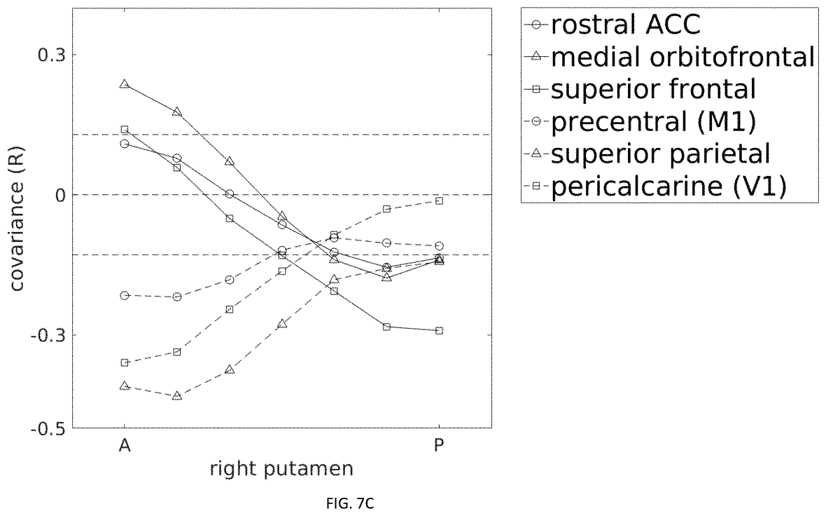

Reference is now made to FIGS. 7A through 7C, which are graphs of experimental measurements, depicting microstructural spatial variations or gradients of cortico-striatal (e.g., between the cortex and striatum) covariation.

FIG. 7A represents semi-quantitative (e.g., T1w/T2w) covariation maps of cortical regions and different subregions along the AP axis of the right putamen. The top part of FIG. 7A schematically represents regions of the cortex, and the bottom part of FIG. 7A schematically represents regions of the putamen. A light-grey marking of the depicted putamen regions indicates the regions for which covariation was calculated. For example, in the left-most image, covariation was calculated for the anterior (A) region of the putamen, and in the right-most image, covariation was calculated for the posterior (P) region of the putamen. Colors of the cortical regions represent the value of covariation between microstructure of the cortical regions and the corresponding region in the putamen. For example, in the left-most pair of images, it may be observed that microstructure of the anterior putamen is (a) strongly, and positively correlated (marked in dark grey) to anterior regions of the cortex, and (b) strongly and negatively correlated (marked in dark grey) to posterior regions of the cortex. In another example, in the right-most pair of images, it may be observed that microstructure of the posterior putamen is weakly, and negatively correlated (marked in light grey) to anterior regions of the cortex.

In this context, the terms covariation and correlation may be used interchangeably to indicate an extent to which a change in one region of the brain (e.g., a putamen) may correspond to a change in another region of the brain (e.g., the cortex).

As shown in FIG. 7A, anterior subregions of the putamen show positive covariance with frontal cortices and negative covariance with parietal and occipital cortices. In the experiment depicted in FIG. 7A, the value of covariation between regions of the putamen and corresponding regions of the cortex may be calculated according to following algorithm: (a) a first gradient quantitative function 130A (e.g., as depicted in FIG. 6C (6C-1, 6C-2)) is calculated along an axis 120A (e.g., AP axis 120A) of the putamen; (b) a second gradient quantitative function 130A (e.g., as depicted in FIG. 6C (6C-1, 6C-2)) is calculated along an axis 120A (e.g., AP axis 120A) of another region of the brain, such as a region of the cortex; and (c) a third gradient quantitative function 130A, representing covariance between the first gradient quantitative function 130A function and the second gradient quantitative function 130A is calculated. The values of this third gradient quantitative function 130A (also referred to herein as a “cortico-striatal covariation”) are depicted as the coloration of cortical regions in FIG. 7A, where dark grey colors represent strong positive or negative covariation, and light grey colors represent weak covariation.

According to some embodiments, microstructure function generator module 130 may be configured to calculate at least one quantitative function 130A that represents a spatial variation or a gradient of cortico-striatal covariation along the AP axis. FIG. 7B depicts an example of 68 such quantitative functions 130A, each corresponding to a specific cortical region. In the example of FIG. 7B, each of the 68 cortical regions is notated with a color that corresponds to its location on the AP axis of the image (y-coordinate of its centroid). As shown in FIG. 7B, profiles of covariation with putamen subregions are distinctively different for frontal (light grey) and posterior (dark grey) cortical regions.

In FIG. 7C, a few quantitative functions 130A were isolated for clarity. As shown in FIG. 7C: (a) there is positive covariation between frontal and limbic regions of the cortex (e.g., the superior frontal region, the medial orbitofrontal region, the rostral anteriorcingulate (rostral ACC) region, etc.), and the anterior putamen; (b) there is negative covariation between motor and sensory cortical regions (e.g., the superior parietal region, the pericalcarine region, the precentral region, etc.) and the anterior putamen; and (c) there is a gradual change (e.g., decrease) of the covariations of (a) and (b) towards the posterior putamen region.

In the experiment depicted in FIGS. 7A through 7C, semi-quantitative (e.g., T1w/T2w) voxel values 210 were used across a large cohort of healthy young adult subjects from the Human Connectome Project (HCP; N=1067, aged 28.8±3.7 years, 606 female).

As elaborated above (e.g., in relation to FIGS. 7A through 7C), experiments have shown a gradient (e.g., a spatial change or variation) of cortico-striatal covariation along the AP axis. While covariation patterns of posterior regions of the dorsal striatum showed little differentiation between cortical areas, anterior regions of the caudate and putamen showed a clear separation between anterior (e.g., rostralanteriorcingulate) and posterior (e.g., superiorparietal) cortical areas. Specifically, anterior subregions of the striatum showed higher positive covariation with frontal and limbic cortices, and negative covariation with more parietal and occipital sensorimotor cortices. This trend was gradually attenuated towards posterior regions of the striatum as shown for example in FIG. 7C.

As shown in the example of FIG. 7C, microstructure of the anterior striatum is adapted to differentiate between cortical regions. In other words, a structural gradient of differential association may be established between the dorsal striatum and the cortex. This observation is consistent with findings regarding selective connectivity of the anterior subregion of the striatum which receive almost no motor or premotor projections, as well as findings of functional selectivity of the anterior striatum toward higher cognitive processing. According to some embodiments, system 100 may exploit this association, and apply this finding to determine, or diagnose a condition of a subject (e.g., a human patient) based on quantitative MRI scans 20.

According to some embodiments, ROI segmentation module 110 may be configured to receive an MRI scan 20 of a tested subject, that may include a plurality of quantitative (e.g., R1) or semi-quantitative (e.g., T1w/T2w) voxel values 210. ROI segmentation module 110 may segment a striatal region 110A from the scan, as elaborated herein, and may also segment a cortical region of interest 110B, in a similar manner.

According to some embodiments, microstructure function generator module 130 may receive (e.g., from SVD module 120) a plurality of quantitative voxel values 210 along an axis 120A of the striatum (e.g., the putamen), as elaborated herein. Microstructure function generator module 130 may further receive a plurality of quantitative voxel values 210 within the cortical ROI 110B. Microstructure function generator module 130 may be configured to calculate at least one quantitative function 130A that is a correlation function, representing correlation between (a) quantitative values of one or more voxels 210 along the at least one axis 120A of the striatum (e.g., putamen) and (b) quantitative values of one or more voxels 210 located in the cortex of the subject. For example, the at least one quantitative function 130A may be a covariation function between frontal and limbic regions of the cortex (e.g., the medialorbitofrontal region) and regions of the putamen, along the AP axis 120A, as depicted in FIG. 7C.

According to some embodiments, analysis module 140 may compare the calculated correlation function to one or more corresponding reference correlation function, that may be stored on a repository database 30. For example, a first reference correlation function may be a covariation function between frontal and limbic regions of the cortex and regions of the putamen, along the AP axis 120A, pertaining to a healthy subject. In another example, a second reference correlation function may be a covariation function between frontal and limbic regions of the cortex and regions of the putamen, along the AP axis 120A, pertaining to a subject who has been diagnosed with a brain-related pathology. According to some embodiments, analysis module 140 may subsequently diagnose a condition of the subject, based on this comparison. For example, analysis module 140 may produce a subject condition notification 50, that may indicate a divergence of quantitative function 130A (e.g., the covariation function) from “normal” or “healthy” values as presented by the first reference correlation function.

It may be appreciated that embodiments of the present invention may not be limited to the example of correlation between the putamen and cortex, as elaborated herein in relation to FIGS. 7A-7C. Instead, correlation between different regions of the brain may also be calculated, and may be compared to predefined reference values, to ascertain a condition of a subject (e.g., a patient). In other words, microstructure function generator may calculate a quantitative function 130A that is a quantitative correlation function 130A. Quantitative correlation function 130A may representing correlation between (a) quantitative, or semi-quantitative (e.g., T1w/T2w) values of one or more voxels along the at least one axis 120A of the striatum and (b) quantitative values or semi-quantitative (e.g., T1w/T2w) of one or more voxels located in another region of the subject's brain (e.g., a region in the cortex, brainstem, amygdala, etc.). Analysis module 140 may compare the calculated quantitative correlation function 130A to a reference quantitative correlation function (e.g., stored in repository 30). Subsequently, analysis module 140 may diagnose a condition of the subject, based on this comparison. Analysis module 140 may also produce a subject condition notification 50 that is indicative of this condition (e.g., includes the result of the comparison), and may send the subject condition notification 50 to a computing device 60 (e.g., a computing device of a physician or care giver).

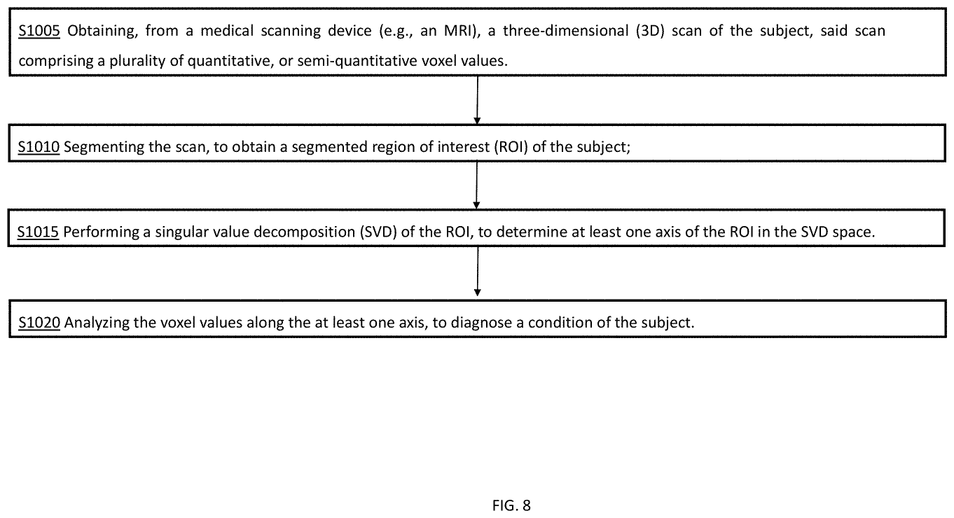

Reference is now made to FIG. 8, which is a flow diagram depicting a method of non-invasive diagnosis of a condition in a subject, by at least one processor, according to some embodiments.

As shown in step S1005, a processor (e.g., element 2 of FIG. 1) of a system 100 for non-invasive diagnosis of a condition in a subject (e.g., a human patient) may obtain, from a medical scanning device (e.g., MRI 11 of FIG. 2), a three-dimensional (3D) scan (e.g., MRI scan 20) of the subject. MRI scan 20 may include a plurality of quantitative or semi-quantitative voxel values 210, as elaborated herein.

As shown in step S1010, processor 2 may utilize a segmentation module (e.g., ROI segmentation module 110 of FIG. 2) to segmenting scan 20, so as to obtain a segmented ROI of the subject. The segmented ROI may, for example include a striatum of a subject, sub striatal organs of the subject, and/or any other regions of the subject's brain.

As shown in step S1015, processor 2 may utilize an SVD module (e.g., SVD module 120 of FIG. 2) to perform a singular value decomposition of the segmented ROI. Processor 2 may thus determine, as known in the art, at least one axis 120A of the ROI in the SVD space.

As shown in step S1020, processor 2 may analyze the voxel values 210 along the at least one axis 120, to diagnose a condition of the subject, as elaborated herein. For example, processor 2 may calculate a quantitative function (e.g., element 130A of FIG. 2) of quantitative, or semi-quantitative voxel values 210 along the at least one axis 120A, and may comparing the calculated quantitative function 130A (e.g., the values of quantitative function 130A) to a reference quantitative function (e.g., to reference values of a reference quantitative function in a repository 30). Processor 2 may determine, diagnosing or predict a condition of the subject, as elaborated herein, based on the comparison. For example, if a value of a calculated quantitative function 130A exceeds, or is below a corresponding reference value of a reference quantitative function, by a predetermined threshold, then processor 2 may determine a condition of the subject, as elaborated in the examples brought herein. Processor 2 may subsequently produce a notification (e.g., element 50 of FIG. 2) of the determined condition, and may transmit this notification to an appropriate computing device 60, as elaborated herein.

Embodiments of the present invention include a practical application for determining, or diagnosing a condition in a subject (e.g., a patient).

As elaborated herein, embodiments of the invention may exploit findings of experimental results, that relate to quantitative or semi-quantitative data pertaining to microstructural variation in the human striatum in vivo. Thus, embodiments of the invention and may include an improvement over currently available methods of diagnosis that are either based on non-quantitative (e.g., morphological) MRI mapping or (b) may only be performed postmortem.

Experimental results have shown that quantitative or semi-quantitative MRI parameters reveal spatial variation or gradient of microstructure along the main axes of the putamen and the caudate nucleus in healthy young adults. Normal aging involves spatially dependent as well as global changes in these gradients. In generalizing the analysis to semi-quantitative MRI contrast, the experimental results uncovered in vivo microstructural markers for Parkinson's disease, that are associated with both dopamine loss quantified by SPECT and disease-related motor function decline, assessed using UPDRS part 3. As elaborated herein, system 100 may exploit these findings to produce a subject condition notification 50 that may include: (a) markers of PD, (b) diagnosis of a subject as suffering from PD, (c) assessment or prognosis of a subject's condition in relation to motor function decline and laterality of motor function decline (d) assessment or prognosis of a subject's condition in relation to dopamine loss, and the like. System 100 may then transmit subject condition notification 50 to a relevant computing device 60, such as a computing device of a physician or a care giver.

Experimental results demonstrated the striatal structure-function relation by showing relationship between striatal microstructure topography and cortical hierarchy. As elaborated herein, system 100 may exploit this finding to produce a subject condition notification 50 that may include, for example, an indication of correlation between the striatal microstructure topography and cortical hierarchy. Embodiments of the invention may then transmit subject condition notification 50 to a relevant computing device 60, such as a computing device of a physician, and may be used (e.g., by the physician) to diagnose or assess a condition of a subject who is suspected to be suffering from a brain-related pathology.

Experimental results have shown existence of in vivo microstructural gradients in the healthy striatum. This finding is consistent with prior works, showing spatial variation or gradients along main axes of the striatum. Animal and postmortem studies found molecular and connectivity gradients in the healthy striatum, using histochemical staining and neural tracing research. Human in vivo research also found gradients of connectivity and function, mostly along the AP axis of the dorsal striatum, using methods of diffusion MRI tractography, resting-state functional MRI (fMRI) and task-based fMRI. Evidence from animal research suggests that developmental gradients of cell migration and signaling in the striatum are what gives rise to the adult healthy neurochemical and connectivity gradients. Additionally, experimental results have shown that aging involves changes in microstructural gradients. In particular, the interhemispheric asymmetry of quantitative voxel values (e.g., R1) gradients in the caudate are enhanced with aging. This finding complements previous studies that showed caudate volume asymmetry increases with aging. As elaborated herein, system 100 may exploit this finding to produce a subject condition notification 50 that may include, for example, an assessment of a tested subject's aging. In other words, system 100 may assess whether the tested subject is experiencing a normal aging process or an excessive aging process. System 100 may then transmit subject condition notification 50 to a relevant computing device 60, such as a computing device of a physician or a care giver.

Experimental results have demonstrated the value of qMRI multiparametric mapping, in studying the biophysical sources of striatal and grey matter variability. R1 is widely considered as a myelin-sensitive measurement. Experimental results have shown that in the striatum, spatial and age dependent R1 variation often correlate with the changes in macromolecular tissue density, quantified by MTV. In addition, experimental results have shown that R2* globally increases with aging. Aging was shown previously to involve R2* increases which are most substantial in the pallidum and the striatum regions, and are correlated with postmortem iron levels in these regions. It is therefore likely that the unique profile of R2* changes found in our analysis, compared to R1, reflects global aging-related increases of iron in the striatum. As elaborated herein, system 100 may exploit this finding to produce a subject condition notification 50 that may include, for example, an assessment of a tested subject's aging. In other words, system 100 may assess whether the tested subject is experiencing a normal aging process or an excessive aging process. System 100 may then transmit subject condition notification 50 to a relevant computing device 60, such as a computing device of a physician or a care giver.

Experimental results have shown irregular spatial variations or gradients of microstructure, showing alterations in posterior subregions of the putamen in subjects suffering from Parkinson's disease. These alterations were manifested as a decrease in the semi-quantitative ratio of T1w/T2w, in comparison to healthy subjects of a control group. Importantly, experimental results have shown that asymmetric decrease of a semi-quantitative (e.g., T1w/T2w) was associated ipsilaterally with asymmetric dopamine loss. This result is consistent with many human and non-human primate studies in PD and parkinsonism, that found spatially-differential tissue alterations, and specifically inhomogeneous dopaminergic degeneration that is most pronounced in posterior parts of the putamen. Furthermore, experimental results show that asymmetric decrease of a semi-quantitative (e.g., T1w/T2w) value in the putamen was contralaterally correlated with motor symptoms' laterality. This finding corroborates previous PET studies that show correlation between Fluorodapa (FDOPA) uptake in the posterior putamen and contralateral motor symptoms severity. In other words, the experimental results provide a non-invasive structural imaging marker for PD laterality in terms of both dopamine loss and motor function decline.

Experimental results have found that cortico-striatal microstructure covariation in healthy individuals demonstrates spatial profiles that correspond to cortical hierarchical organization. These results show that anterior subregions of the dorsal striatum show positive covariation with frontal associative and limbic regions on the one hand, and a negative covariation with sensorimotor parietal and occipital regions on the other hand. This observation may suggest differential involvement of striatal subparts in different neural domains. This agrees with prior works relating striatal subregions to differential cortical connectivity and co-activity.

As elaborated herein, system 100 may use the semi-quantitative (e.g., T1w/T2w) contrast or values to analyze and determine PD-related changes in striatal microstructure. Several studies previously established T1w/T2w sensitivity to cortical microstructure. Experimental results have shown that standardized T1w/T2w spatial variation in the striatum is to a large extent similar to that of quantitative R1. This may suggest that PD-related changes in semi-quantitative (e.g., T1w/T2w) values reflect general atrophies of the striatal tissue, that may be a consequence of depletions in dopaminergic innervation.

Currently available methods for analysis of qMRI scans may calculate changes in qMRI parameters along white-matter tracts and across cortical layers. In contrast, embodiments of the invention may include automatic quantification of qMRI spatial variation along axes 120A of subcortical grey matter structures (e.g., the dorsal striatum) within the subject space. By extracting axes 120A (e.g., by SVD module 120, as elaborated herein), embodiments of the invention may improve robustness and repeatability of the analysis process, in relation to currently available methods.

Examples of application of the present invention as elaborated herein mainly relate to the dorsal striatum. However, it may be appreciated by a person skilled in the art that applications of embodiments of the invention are not limited to the dorsal striatum, and may be used for other heterogeneous subcortical structures, such as the pallidum and brainstem regions.

Additionally, it may be worth noting that sampling of microstructural change along the main orthogonal axes 120A of the subcortical structure is somewhat arbitrary. It is highly plausible that the first component of microstructural variance may not aligned with one of the three orthogonal axes 120A defined by the region's morphology. It may be appreciated that additional applications may include sampling of microstructural change along other axes 120A of the subcortical structure, with the required modifications, depending on the application.

Embodiments of the invention we propose a non-invasive, in-vivo approach to quantify microstructural spatial variation or gradients in the human striatum, using quantitative MRI. This approach may include a unique insight on local and global aging and disease-related changes in the striatal tissue. Embodiments of the invention may use different qMRI parameters to find global and spatially-dependent changes in the aging of the caudate and putamen. Moreover, embodiments of the invention may detect local alterations in the putamen in pathological cases such as PD, which are related to dopaminergic loss and to motor function decline.

Embodiments of the invention may be used to correlate microstructure variation with (a) biophysical sources and/or (b) motor function.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of non-invasive diagnosis of a condition in a subject by at least one processor, the method comprising:
obtaining, from a medical scanning device, a three-dimensional (3D) scan of the subject, said scan comprising a plurality of voxel values;
segmenting the scan, to obtain a segmented region of interest (ROI) of the subject;
performing a singular value decomposition (SVD) of the ROI, to determine at least one axis of the ROI in the SVD space; and
analyzing the voxel values along the at least one axis, to diagnose a condition of the subject.

2. The method of claim 1, wherein analyzing the voxel values along the at least one axis comprises:
calculating a quantitative function of voxel values along the at least one axis;
comparing the calculated quantitative function to a reference quantitative function; and
diagnosing a condition of the subject, based on said comparison.

3. The method of claim 2, wherein the medical scanning device is a magnetic resonance imaging (MRI) scanning device, and wherein the voxel values are quantitative, or semi-quantitative voxel values, selected from a list consisting of R1, R2, T1, T2, T1w/T2w, proton density and macromolecular tissue volume (MTV) values, diffusion parameter values selected from MD, FA, QSM and CEST values, and any combination thereof.

4. The method of claim 2, wherein the medical scanning device is an MRI scanning device, and wherein scanning the subject comprises:
obtaining a first, weighted T1 scan of the subject, comprising a first plurality of voxel values;
obtaining a second, weighted T2 scan of the subject, comprising a second plurality of voxel values; and
elementwise dividing the first plurality of voxel values by the second plurality of voxel values, to obtain a semi-quantitative voxel values.

5. The method of claim 2, wherein the segmented ROI comprises a putamen of the subject, and wherein the quantitative function represents a spatial variation of voxel values along the at least one axis, through the putamen.

6. The method of claim 2, wherein the segmented ROI comprises a caudate of the subject, and wherein the quantitative function represents a spatial variation of voxel values along the at least one axis, through the caudate.

7. The method of claim 1, wherein analyzing the voxel values along the at least one axis further comprises:
calculating a quantitative correlation function, representing correlation between (a) quantitative values of one or more voxels along the at least one axis of the ROI and (b) quantitative values of one or more voxels located in another region of the subject's brain;
comparing the calculated correlation function to a reference correlation function; and
diagnosing a condition of the subject, based on said comparison.

8. The method of claim 2, further comprising predicting a condition of dopaminergic loss in the subject based on said comparison.

9. The method of claim 2, wherein the quantitative function represents a metric of asymmetry between (a) quantitative voxel values along at least one axis of a left hemisphere striatum and (b) quantitative voxel values along at least one axis of a right hemisphere striatum, and wherein the method further comprises:
comparing the quantitative function to a reference quantitative function; and
predicting a condition of dopaminergic loss in the subject based on said comparison.

10. The method of claim 2, further comprising predicting a condition of motor function decline in the subject based on said comparison.

11. The method of claim 2, wherein the quantitative function represents a metric of asymmetry between (a) quantitative voxel values along at least one axis of a left hemisphere
striatum and (h) quantitative voxel values along at least one axis of a right hemisphere striatum, and wherein the method further comprises:
comparing the quantitative function to a reference quantitative function; and
predicting a condition of motor function decline in the subject based on said comparison.

12. A system for non-invasive diagnosis of a condition in a subject, the system comprising:
a non-transitory memory device, wherein modules of instruction code are stored, and a processor associated with the memory device, and configured to execute the modules of instruction code, whereupon execution of said modules of instruction code, the processor is configured to:
obtain, from a medical scanning device, a three-dimensional (3D) scan of the subject, said scan comprising a plurality of voxel values;
segment the scan, to obtain a segmented region of interest (ROI) of the subject;
perform a singular value decomposition (SVD) of the ROI, to determine at least one axis of the ROI in the SVD space; and
analyze the voxel values along the at least one axis, to diagnose a condition of the subject.

13. The system of claim 12, wherein the processor is configured analyze the voxel values along the at least one axis by:
calculating a quantitative function of voxel values along the at least one axis;
comparing the calculated quantitative function to a reference quantitative function; and
diagnosing or predicting a condition of the subject, based on said comparison.

14. The system of claim 13, wherein the segmented ROI comprises a putamen of the subject, and wherein the quantitative function represents a spatial variation of voxel values along the at least one axis, through the putamen.

15. The system of claim 13, wherein the segmented ROI comprises a caudate of the subject, and wherein the quantitative function represents a spatial variation of voxel values along the at least one axis, through the caudate.

16. The system of claim 12, wherein the processor is configured to analyze the voxel values along the at least one axis by:
calculating a quantitative correlation function, representing correlation between (a) quantitative values of one or more voxels along the at least one axis of the ROI and (b) quantitative values of one or more voxels located in another region of the subject's brain;
comparing the calculated correlation function to a reference correlation function; and
diagnosing a condition of the subject, based on said comparison.

17. The system of claim 13, wherein the processor is further configured to predict a condition of dopaminergic loss in the subject based on said comparison.

18. The system of claim 13, wherein the quantitative function represents a metric of asymmetry between (a) quantitative voxel values along at least one axis of a left hemisphere striatum and (b) quantitative voxel values along at least one axis of a right hemisphere striatum, and wherein the processor is further configured to:
compare the quantitative function to a reference quantitative function; and
predict a condition of dopaminergic loss in the subject based on said comparison.

19. The system of claim 13, wherein the processor is further configured to predict a condition of motor function decline in the subject based on said comparison.

20. The system of claim 13, wherein the quantitative function represents a metric of asymmetry between (a) quantitative voxel values along at least one axis of a left hemisphere striatum and (b) quantitative voxel values along at least one axis of a right hemisphere striatum, and wherein the processor is further configured to:
compare the quantitative function to a reference quantitative function; and
predict a condition of motor function decline in the subject based on said comparison.
